SEQUENCE LISTING (1 sequence)

SEQ ID NO: 1 - DNA, 820 bp, Streptomyces rutgersensis 88
  misc_feature (1)...(820): PreSequenceString
natgaagcccttcggggtggattagtggcgaacgggtgagtaacacgtgggcaatctgccctgcactctgggacaagccctggaaacggggtctaataccggatatgaccgtccatcgcatggtggatggtgtaaagctccggcggtgcaggatgagcccgcggcctatcagctagttggtgaggtagtggctcaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggttgtgaaagcccggggcttaaccccgggtctgcagtcgatacgggcaggctagagttcggtaggggagatcggaattcctggtgtagcgntgaaatgcgcagatatcaggaggaacaccggtggcgaangcgganctctgggccgatactgacgctgaggagcgaaagcgtggggagcgaacaggantagataccctgnnagtncacgncgtanacggngggcactaggngtgggcaacattccacgttgtccgtgccnnanctaanncnntaannnncccnccnggggannnnggncnnaaggntaanntcan

(19) United States
(12) Patent Application Publication
(10) Pub. No.: US 2016/0184343 A1
(43) Pub. Date: Jun. 30, 2016

(54) THERAPEUTICAL BETA-GLUCAN COMPOSITION, MODULATING HUMAN IMMUNE SYSTEM AND INITIATING THE BREAKDOWN OF CANCEROUS CELLS

(71) Applicant: UAB "BIOCENTRAS", Vilnius (LI)

(72) Inventors: Saulius Grigiskis, Vilnius (LT); Artur Javmen, Vilnius (LT); Mykolas Mauricas, Vilnius (LT); Vilma CIPINYTE, Vilnius (LT); Darius KACANAUSKAS, Vilnius (LT); Fortunatas Grygas, Vilnius (LT); Julius VAITOSKA, Vilnius (LT)

(21) Appl. No.: 14/435,227

(22) PCT Filed: May 23, 2014
(86) PCT No.: PCT/IB2014/061654

§ 371 Date: Apr. 13, 2015

(30) Foreign Application Priority Data
Apr. 14, 2014 (LT) 2014060

(51) Int. Cl. A61K 31/716; C12P 19/04; A61K 45/06; C12P 19/14

(52) U.S. Cl. CPC A61K 31/716; C12P 19/14; C12P 19/04; A61K 45/06

(57) ABSTRACT

This invention relates to the field of biotechnology. It describes organism immunostimulation, initiation of breakdown processes of cancerous cells and prevention of the formation of metastases, using water soluble β-glucan compositions of various molecular masses. In this invention is also described unique biocatalyst produced by Streptomyces rutgersensis 88 and used to obtain β-glucan mixtures, is also described.

### TECHNICAL FIELD

This invention relates to the field of biotechnology. It describes organism immunostimulation, initiation of breakdown processes of cancerous cells and prevention of the formation of metastases, using water soluble β-glucan compositions of various molecular masses. Unique biocatalyst produced by Streptomyces rutgersensis 88 and used to obtain β-glucan mixtures, is also described in this invention.

### BACKGROUND OF INVENTION

Scientific research determined that different polysaccharides composed of repeating glucose monomers, such as cellulose, starch, chitin, mannan and others, are the most prevalent polymers on the planet. One of such glucose polymers is β-glucan, which is composed of glucose monomers linked by β-glycosidic bonds. There are various types of β-glucans that are produced by different organisms—bacteria, fungi, plants—in the nature. Most frequently β-glucans are found within organism cell walls (Javmen et al., 2013; Basic et al., 2009). Different β-glucans can have different structure. That depends on glycosidic bonds that join the glucose monomers; composition's physical properties depend on polymer branching. For example, curdlan is a linear Alcaligenes faecalis β-(1-3) water soluble β-glucan and cellulose is a complex water insoluble β-(1-4) glucan (Javmen et al., 2012; Basic et al., 2009; Novak et al., 2008; Peliosi et al., 2006). It was discovered that noncellulosic β-glucans of fungal origin irritate mammalian immune system and can serve as potential human immune system stimulants (Chen et al., 2007). Due to this reason β-glucans of fungal origin are widely researched worldwide. Experiment data shows that these β-glucans protect mammals from various infections and increase immune system cells' cytotoxicity towards cancerous cells (Chang et al., 2009; Vetvicka, 2011).

As it was mentioned, β-glucans are produced by various organisms, thus those compounds are extracted from different sources. Hereafter, a few examples of such sources are presented. U.S. Pat. No. 6,660,722 B2 describes the application of β-glucan laminarin, which is isolated from microorganism Laminaria saccharina. Patent WO03/045414 uses laminarin isolated from Laminaria digitata. Patent US 2007/0117777 A1 uses β-glucan isolated from Aureobasidium pullulans. Patent US 2008/0311243 A1 uses β-glucan isolated from grains. Patent EP1361264 B1 uses β-glucan isolated from barley. Patent US 2009/0098619 A1 describes the production of β-glucan using various types of fungi, e.g. Ganoderma lucidum, Coriolus versocolor and different species of Lentula fungi. An important source of immunoactive β-glucans is baking yeast Saccharomyces cerevisiae (Javmen et al., 2012; Basic et al., 2009; Novak et al., 2008; Hunter et al., 2002). The use of β-glucan obtained from Saccharomyces cerevisiae is described within the following patents: U.S. Pat. No. 5,622,939, US 2006/0247205 A1, U.S. Pat. No. 4,810,646, U.S. Pat. No. 7,550,584 B2, WO 2006/119395 A2, WO 2004/082691 A1, WO 1997/002356 A1, US 2013/0310338 A1, WO92/13896, U.S. Pat. No. 8,323,644 B2. A large part of yeast mass is composed of β-glucans, as they are one of the main cell wall components. Yeast cell wall is formed of two layers—inner and outer. Outer layer contacts with a medium on the exterior of the cell and is composed of mannoproteins and proteins, while the inner cell layer is formed of many different polysaccharides. The largest percentage of inner polysaccharide layer is composed of β-glucan, which forms the layer skeleton. Other polysaccharides are also covalently attached to β-glucan (Klis et al., 2006). The base of yeast cell inner layer β-glucan is composed of long glucose polymer chains linked with β-1,3 bonds, which are additionally linked between themselves with β-1,6 bonds. Thus, those structures form tridimensional polymer network on the exterior of the protoplast (Bacic et al., 2009). The average length of separate β-glucan chains is ˜600 nm and they are formed of ˜1500 glucose monomers (2.4×10⁵ Da) (Bacic et al., 2009).

Different methods are used for the separation of β-glucan from yeast Saccharomyces cerivisiae. The following procedures need to be performed in order to separate insoluble β-glucan from the yeast cells of Saccharomyces cerevisiae:
1. Breaking down the yeast cells and separating insoluble cell walls from liquid cytoplasm (as β-glucan is localized within cell walls);
2. Isolating an insoluble β-glucan from the insoluble cell walls' precipitate.

Many methods can be applied in order to lyse yeast cells; they can be separated into three categories: a) chemical, b) physical, c) enzymatic. Sodium hydroxide, hydrochloric acid, acetic acid and other aggressive chemical solutions can be used for chemical cell lysis (Pelizon et al., 2005; Zechner-Krpan et al., 2010). Also, physical methods can be used for yeast cell breakdown: ultrasound and homogenizer application (Shokri et al., 2008). One more group of methods used for the yeast cell breakdown is enzymatic methods, which respectively can be separated into two sub-groups:
a) yeast cell autolysis—cell is lysed by its own enzymes associated with cell death (Martinez-Rodriguez et al., 2001). Yeast cell autolysis is carried out at a temperature of >50° C., when there are no nutrients in the medium. Process is long and can last for days (Vosti et al., 1954; Hernawan, Fleet 1994).
b) the use of yeast lysing enzymes synthesized by other organisms. There is not a small amount of microorganisms secreting the enzymes that lyse yeast cells into their surroundings. These enzymes can be used for yeast cell breakdown processes (Gilbert et al., 2002).

After the breakdown of yeast cells, insoluble β-glucan is isolated from insoluble yeast cell precipitate. Alkalis, various acids, hydrogen peroxide and etc. are usually used to that end. Majority of materials composing cell walls pass into a liquid state after having been affected by alkalis and acids. β-glucan is insoluble in alkali even at high concentrations, thus it is most frequently used for β-glucan separation from other yeast cell wall polymers (Jamas et al., 1989; Hayen et al., 2001; Shokri et al., 2008; Bacic et al., 2009; Bahl et al., 2009). Hereinafter, some of the methods for β-glucan separation from S. cerevisiae, as described in various patents, are presented. U.S. Pat. No. 7,550,584 B2, U.S. Pat. No. 4,810,646, WO 2004/082691 A1, U.S. Pat. No. 5,622,939, U.S. Pat. No. 7,776,843 B2, WO 2007/146416 describe a method based on treating S. cerevisiae with alkali and after that with acid. Patent US 2013/0310338 A1 describes a method based on treating S. cerevisiae with alkali and after that with ethanol and water. Patent WO92/13896 presents a method for treating S. cerevisiae with DMSO and after that with sulfuric acid.

It is known that the effect of β-glucans on immune system depends on the size of β-glucan molecules (Mantovani et al., 2008). The recognition of β-glucans of fungal origin is enacted through special receptors, which are localized on the surface of leucocytes (macrophages, neutrophils, NK cells and etc.). It has been determined that β-glucan interaction strength with different receptors depends on the size of β-glucan molecules (Akramiene et al., 2007; Mantovani et al., 2008). The main receptors recognizing β-glucan molecules are complement receptor CR3, Dectin-1, also called β-glucan receptor, Toll-like receptor TLR-2, lactosylceramide and some other receptors (Akramiene et al., 2007; Novak et al. 2008; Chen et al., 2007).

General immunostimulation. It is known that β-glucans stimulate the immune system in order to fight against infectious diseases and cancer. β-glucans can cause cytokine synthesis, improve microbe phagocytosis and breakdown properties, after being subjected to the influence of macrophages, neutrophils, NK and dendritic cells, and other leucocytes possessing β-glucan recognition receptors (Akramiene et al., 2007; Novak et al., 2008; Chen et al., 2007, Vetvicka et al., 2012). Macrophages that are activated by β-glucan molecules can act not only against antigen-containing β-glucanes, but against any other antigens, like pathogenic microorganisms, or even against cancerous cells. Macrophage cells are the only ones that can phagocyte β-glucans and oxidize them into compounds of simpler structure. It is relative to mammals not having β-glucanase enzymes and thus it is the only way to remove β-glucans from mammalian organisms (Novak et al., 2008).

Anti-cancer effect. It is known that β-glucan can stimulate the immune system in order to fight against cancerous cells (Akramiene et al., 2007; Novak et al., 2008; Chen et al., 2007, Vetvicka et al., 2012). The actual β-glucan operation mechanism is not clear (Vetvicka et al., 2012). It has been determined that after being influenced by β-glucan molecules neutrophils, macrophages, eosinophils and NK cells can breakdown cancerous cells using CR3-DCC (cytotoxicity dependent on complement receptor 3) mechanism, in which they usually do not participate (Vetvicka et al., 2012, Yan et al., 2009). It has been proven that insoluble β-glucan in mammal intestines is phagocyted by intestinal macrophages and then is transported to spleen, lymphatic nodes and bone marrow. Macrophages degrade large insoluble β-glucan particles into smaller soluble fragments within bone marrow (Yan et al., 2009; Chan et al., 2009). These smaller β-glucan molecules can interact with leucocyte receptor's CR3 lectin part (neutrophils, macrophages, eosinophils and NK cells) (Yan et al., 2009; Chan et al., 2009; Vetvicka et al., 2012). In many cases, tumour cells are opsonized with antibodies and complement protein iC3b (Vetvicka et al., 2012). CR3 receptor in neutrophils, macrophages, eosinophils and NK cells recognizes such opsonized cancerous cells and, after an interaction with small soluble β-glucan cells, can break them down; though, as it was mentioned, usually these leucocytes do not degrade cancerous cells according to this mechanism (Yan et al., 2009; Chan et al., 2009; Vetvicka et al., 2012). Normal, non-mutated tissue cells are protected from the leucocyte attack, because they are not opsonized with complement protein iC3b (Vetvicka et al., 2012).

A few cancer therapy methods based on such knowledge were patented. U.S. Pat. No. 6,660,722 B2 describes a cancer treatment method that uses soluble β-glucan laminarin for tumour breakdown (the example describes laminarin from Laminaria saccharina). Patent WO 2004/021994 A2 describes cancer treatment method based on the use of insoluble particular β-glucans from oats, fungi or yeast (Saccharomyces cerevisiae and other species) together with antibodies activating complement system against cancerous tumour or cancer antigens. Patent WO 2005/049044 A1 describes cancer treatment method that uses synthetic β-glucan oligomers (up to 10 monomers; optimally 2-3) together with monoclonal antibodies against determinants on the surfaces of cancerous cells. The authors of patent US 2009/0074761 A1 have patented therapeutic β-glucan combinations, which will be used as medicine against cancer. Patented therapeutic blends are composed of different β-glucans (soluble and insoluble) and antibodies—VEGF antagonists. In this case, β-glucan is isolated from yeast. U.S. Pat. No. 8,323,644 B2 describes a method to treat cancer with a blend of anti-cancer antibodies and β-glucan isolated from yeast S. cerevisiae; β-glucan can be in a soluble or insoluble form. Patent WO 2004/082691 describes the production of soluble S. cerevisiae β-glucan, which is used for cancer treatment and prevention.

β-glucan can also be used to treat other disorders. U.S. Pat. No. 7,776,843 describes the treatment of broken bones using yeast β-glucan. Patent WO 2007/144616 describes a use of yeast S. cerevisiae β-glucan for general immunostimulation.

U.S. Pat. No. 8,367,641 B2 describes the synthesis of modified β-glucans, which can be used to break down tumours, treat viral, bacterial and fungal infections and immune system disorders. Patent US 2008/0167268 describes the method for activating cytokine synthesis using particular S. cerevisiae β-glucan. U.S. Pat. No. 8,318,218 B2 describes weight reduction method using S. cerevisiae β-glucan. Patent US 2007/0117777 A1 describes osteoporosis prevention and treatment method, which uses β-glucan from Aurobasidium pullulans for breaking down tumours.

Patent source analysis shows us that biomedical research was performed in two directions:
the search for new β-glucan sources;
the application of β-glucans for medical therapy.

Considering different β-glucan isolation methods and their primary sources, their therapeutical effect is also different. Some β-glucans are applied for immunomodulation; others are more or less effectively used for the inhibition of pathogenic microorganisms and degradation of cancerous cells.

Macrophage activation is the response of immune system to large molecular mass water soluble β-glucans. They activate the immune system and increase the amount of dendritic cells in blood. Meanwhile, smaller molecular mass water soluble β-glucans are like the second recognition signal of the cancerous cells according to the CR3-DCC mechanism. It allows us to use them as initiators for the breakdown-killing of cancerous cells.

Cancerous cells can quickly adapt to damaging external factors. Cancerous cells which are dead due to the effects of chemotherapy or radiation can poison the organism. This reason is why the direct degradation of cancerous cells is in many cases complicated, as a lot of toxic compounds enter the blood stream. That makes it virtually impossible to prevent the formation of metastases and the breakdown of healthy cells. In short, these therapeutic methods are not selective regarding cancerous and healthy cells. The breakdown products of dead cells encumber the transport of nutrient materials into the cells and thus cancerous cells migrate to other places in the organism, creating metastases.

The foregoing methods for organism immunostimulation and breakdown of cancerous cells do not fully solve all the problems associated with cancer therapy:
cancer therapy is a complex process, requiring high qualification human resources and technologies;
there is no universal complex preparations for both immunomodulation and the initiation of breakdown of cancerous cells;
there is no preparations and methods for the regulation of cancerous cells' breakdown speed;
there is no synthetic or biological preparations that can stop the spreading of metastases;
there is no complex preparations that can prevent the cancerous cells from adapting to them.

### SUMMARY OF THE INVENTION

The goal of this invention is to create a therapeutical composition of β-glucans, modulating human immune system and initiating the breakdown processes of cancerous cells.

The essence of this invention is a β-glucan composition, composed of water soluble β-glucans of various molecular mass, capable of both immunomodulation and the initiation of breakdown processes of cancerous cells.

β-glucans of large molecular mass activate phagocytes, promote the synthesis of γ-interferon, hence stimulating organism immune system and breaking down single cancerous cells.

Water soluble β-glucans of small molecular mass interact with neutrophil CR3 receptors initiating the breakdown of all cancerous cells. In order the breakdown of cancerous cells to be performed in an optimal way and not cause the termination of an organism, it's important to retain the ratio of the small and large molecular mass β-glucans within their composition.

In order to control the breakdown of cancerous cells, the compositions with changeable different molecular mass β-glucan concentrations were used.

As to prevent the formation of metastases, β-glucan composition with immunomodulating and the breakdown of cancerous cells initiating β-glucan components of fixed concentrations, is prepared.

The method for the breakdown of cancerous cells, described in this invention, is different from other known ones, as insoluble β-glucan is isolated from Sacharomyces cerevisiae after specific enzymatic hydrolysis, and therapeutical compositions are prepared from obtained water soluble β-glucans.

The second difference is that soluble β-glucans are obtained through specific enzymatic hydrolysis, in the presence of purified β-1,3-glucanase from Streptomyces rutgersensis 88.

The third difference is that β-glucans are specifically hydrolyzed to oligosaccharides of a certain molecular mass, in the presence of β-1,3-glucanase from Streptomyces rutgersensis 88.

The fourth difference is that soluble β-glucans are fractioned into immunomodulators and agents initiating the breakdown of cancerous cells.

The fifth difference is that, in order to increase the efficacy of breakdown of cancerous cells process, β-glucan enzymatic hydrolysis fractions of various molecular mass, and whose intrinsic viscosity is in a range of 0.3 to 0.01, preferably is from 0.08 to 0.02, are joined together.

The sixth difference is that in order to prevent the adaptation protective systems of cancerous cells, agents of different molecular mass, initiating the breakdown of cancerous cells, are used.

The seventh difference is that during the breakdown of cancerous cells, organism immunomodulation is the first to be performed, and after that cancerous cells are marked with breakdown initiating agent.

The eighth difference is that immunostimulating biopreparations and agents initiating breakdown of cancerous cells are introduced alternately.

The ninth difference is that the order for introducing immunostimulating biopreparations and agents initiating breakdown of cancerous cells into organism is alternating until cancerous cells are degraded.

The tenth difference is that immunostimulators and agents initiating breakdown of cancerous cells are introduced into organism in vitro, gradually increasing their concentrations, in order to prevent damage to healthy organism cells.

The eleventh difference is that the breakdown process of cancerous cells is artificially inhibited, keeping low concentrations of agents initiating breakdown of cancerous cells and high immunostimulation, so that the tissue composed of cancerous cells would break down slowly and the poisoning of the organism with products of toxic cancerous cells would be prevented.

The twelfth difference is that the concentrations of immunostimulators and agents initiating breakdown of cancerous cells are changed from 0 to 1000 mg/ml until the breakdown process is fully completed.

The thirteenth difference is that in order to prevent the growth of secondary cancerous cells (metastases), the breakdown process is repeated while keeping the concentrations of immunostimulators and agents initiating breakdown of cancerous cells sufficient to degrade cancerous cells.

The fourteenth difference is that in order to prevent the adaptation of cancerous cells to the components of active immune system that break them down, water soluble β-glucans of various molecular mass are used for the immunorecognition of cancerous cells.

### BRIEF DESCRIPTION OF THE DRAWINGS

FIG. 1. Preparation of Master and Working cell banks of S. rutgersensis 88 culture (stage I)

FIG. 2. Biosynthesis of yeast lysing complex by Streptomyces rutgersensis 88 strain (stage II)

FIG. 3. Chromatographic purification of β-1,3-glucanase enzyme (stage III)

FIG. 4. Preparing of water insoluble β-glucan preparation (stage IV)

FIG. 5. Preparing of water soluble β-glucan preparations (stage V)

FIG. 6. Dependency of fractions after chromatographic separation and joined fractions on total carbohydrate amount

FIG. 7. Thin-layer chromatograms before fractionation process and after it: a) before chromatography; b) after chromatography (fractions 1, 2, 3, 4, 5, 6)

FIG. 8. Dependency of intrinsic viscosity on molar masses of fraction components

FIG. 9. Principal working scheme of therapeutical β-glucan compositions

FIG. 10. Influence of β-glucan preparations on the increase of IFN-γ expression in vivo

FIG. 11. The amount of dead cancerous cells (in percents) after the treatment with β-glucan preparation compositions in vitro

### DESCRIPTION OF PREFERRED EMBODIMENTS

Commercial bakers' yeast Saccharomyces cerevisiae strain number HEM: 7071, are used for the production of water insoluble β-glucan and water soluble β-glucan preparations.

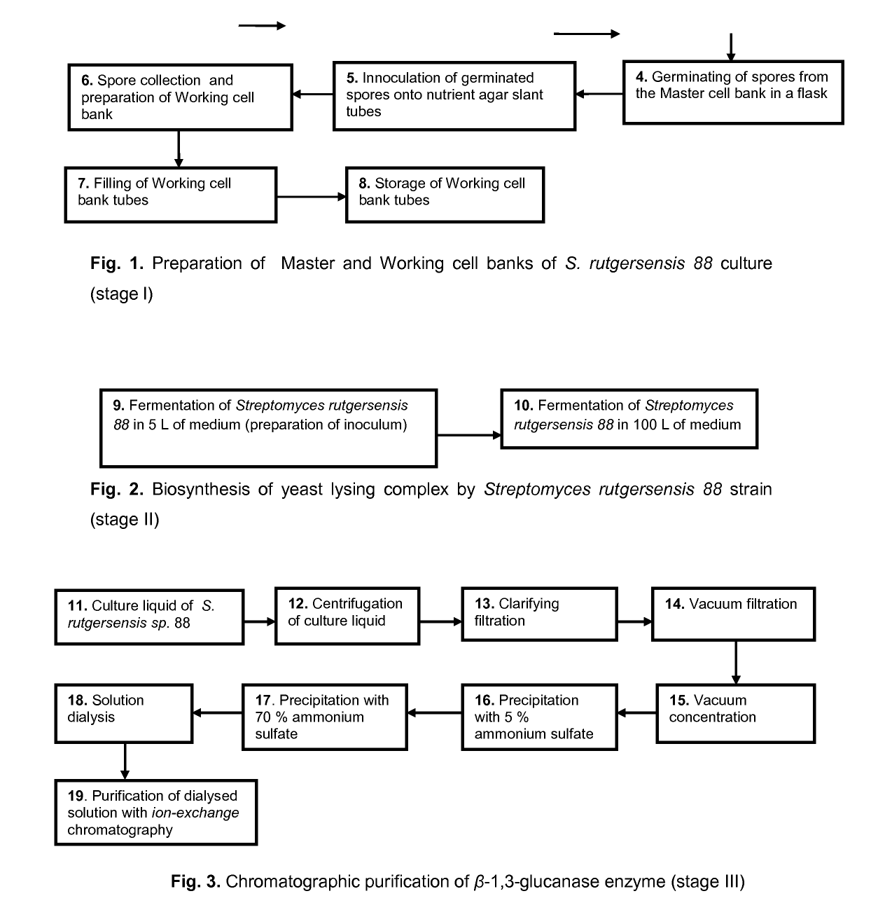

Lysing complex obtained from Streptomyces rutgersensis 88 is used for the separation of these preparations from yeast cell walls and their hydrolysis. Firstly, the selection of Streptomyces rutgersensis 88 is performed and Master and working cell banks are prepared, in order to obtain a complex with optimal yeast lysing and β-glucanasic activities (FIG. 1).

Further, the characteristics of the microorganism producing the said lysing complex are presented.

Streptomyces rutgersensis 88 strain (JSC “Biocentras” accession No. K-91-2) was isolated from soil in Lithuania.

Cells. Cells are gram-positive, create branched hyphae with a diameter of 0.4-0.8 μm. After the maturation of culture, the chains of oval or oblong spores are formed on the ends of hyphae. Spores are nonmotile, their surface is smooth.

Cultural properties. Abundant mycelium (aerial mycelium: at first brown, after maturation spores are grown and mycelium turns brownish-greyish-white; substrate mycelium: brown) forms while growing culture on a solid maize agar No. 2. Whitish aerial and brownish substrate mycelium is formed on soy-peptone agar.

Physiological-biochemical properties. It's an aerobe. Catalase reaction is positive. Optimal growth conditions: temperature 28-30° C., pH 7.0-7.8. Culture is negative for acid-fast staining and hydrolyses casein, gelatin, β-glucans, doesn't hydrolyse starch, lyses yeast and bacteria cell walls. Uses glucose, arabinose, xylose, fructose, mannite. Doesn't use inositol, saccharose, rhamnose and raffinose.

Based on 16S rRNA gene sequence shown in SEQ ID No. 1, this microorganism is closest to species Streptomyces rutgersensis.

In order to ascertain an uninterrupted production of β-glucan preparations, a larger amount of yeast lysing and water insoluble β-glucan hydrolysing complex is prepared (FIG. 2).

Streptomyces rutgersensis 88 culture liquid obtained after the biosynthesis of yeast lysing complex is used for the separation of water insoluble β-glucan from yeast cell walls. Before the hydrolysis of water insoluble β-glucan, β-1,3-glucanase is purified chromatographically from culture liquid, so that final water soluble β-glucan preparations would have the least amount of impurities (FIG. 3).

In order to obtain β-glucan preparations, firstly, insoluble β-glucan is separated from yeast cell walls, using yeast lysing complex and 2 M NaOH solution in water (FIG. 4).

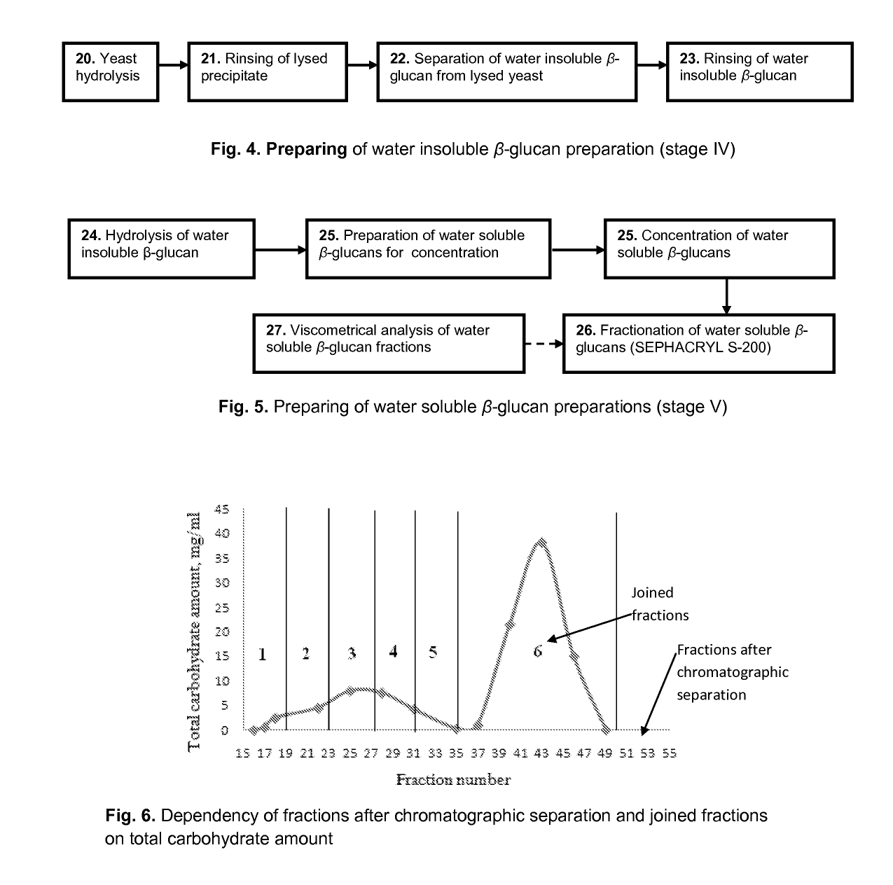

Purified water insoluble β-glucan is specifically hydrolysed, using chromatographically purified Streptomyces rutgersensis 88 yeast lysing complex with β-glucanase activity (FIG. 5). Obtained water soluble hydrolysate is separated into water soluble β-glucan fractions by gel chromatography. Fractionation process is additionally standardized viscometrically measuring intrinsic viscosity of water soluble β-glucan fractions.

Hereunder, a few examples of invention method application are described.

Fractionation of Water Soluble β-Glucans

Water insoluble β-glucan obtained from baking yeast Saacharomyces cerevisiae is used as a substrate for the production of water soluble β-glucan preparations. Streptomyces rutgersenisis 88 enzyme β-1,3-glucanase is used for the hydrolysis of water insoluble β-glucan. A mixture of soluble β-glucan molecules, obtained following the hydrolysis of water insoluble β-glucan, is concentrated. Obtained concentrate, composed of β-glucans of various molar mass, is separated into 5 fractions using gel chromatography.

Concentrated mixture of water soluble β-glucans is placed into a gel chromatography column filled with SEPHACRYL S-200 sorbent. A fixed quantity of fractions is collected during the gel chromatography process. Mobile chromatography phase is 0.01 M potassium phosphate buffer solution with a pH value of 7. A volume of introduced β-glucan mixture is no more than 5% of total column volume, preferred is no more than 3%. Carbohydrate concentration is determined in the fractions collected after the chromatographic separation.

Fractions, having the highest carbohydrate concentration and composed mostly of small glucose oligomers (usually the final fractions), are joined into a single fraction after the end of the fractionation process. Other fractions are joined into 5 larger fractions of the same volume in sequential order, i.e. from the lowest fraction number to the highest. Joined fractions are analyzed by these methods: thin-layer chromatography and by measuring their intrinsic viscosities.

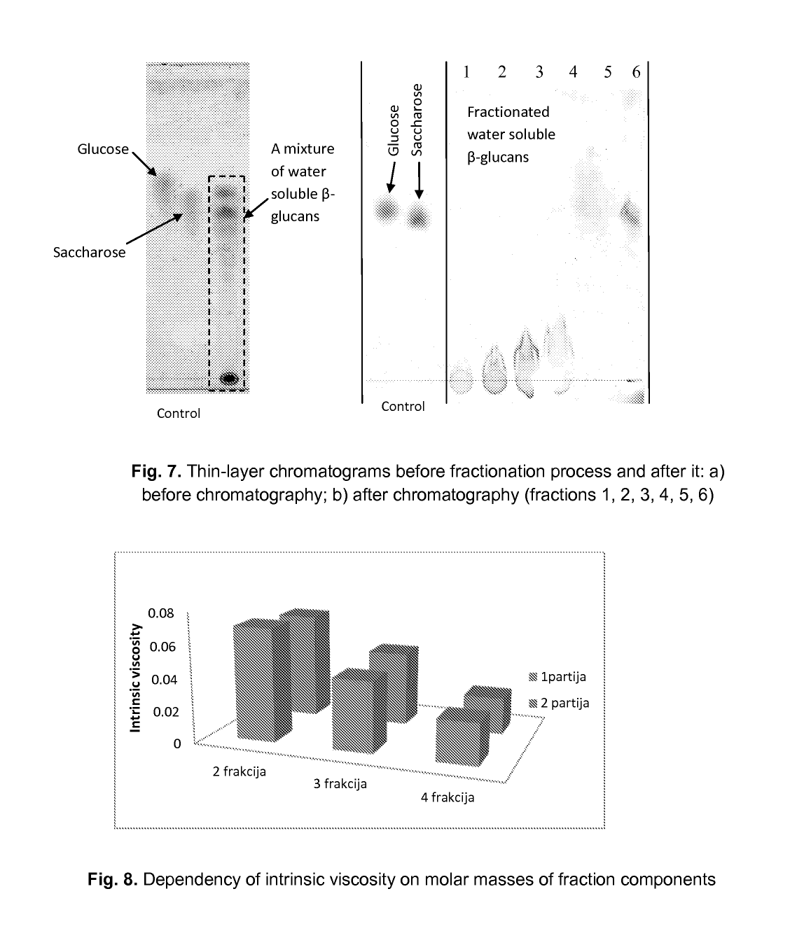

Typical chromatograms of water soluble β-glucan during fractionation process (carbohydrate concentration) are shown in FIG. 6 and FIG. 7.

Determination of Intrinsic Viscosity of Chromatographically Separated Water Soluble β-Glucans using Capillary Viscometer

Six water soluble β-glucan fractions were obtained using molecular sieve chromatography. After that, flow times are measured and intrinsic viscosities are calculated in every fraction using capillary viscometer. Procedure is repeated 4-5 times and obtained data is used to calculate the flow time arithmetical average. The data is used to graphically determine reduced viscosity and intrinsic viscosities. Intrinsic viscosity is determined by extrapolating of reduced viscosity to an infinitesimal concentration. A line drawn through the obtained points severs the value of intrinsic viscosity on the ordinate axis.

Intrinsic viscosity is compared among the same β-glucan solution fractions, if their reduced viscosities are measured in uniform conditions. Reduced viscosity is measured at the temperature of 25° C. with the same solution volumes and the same concentrations.

Viscosity of diluted β-glucan solutions is directly related to the size and conformation of β-glucan macromolecules. Specific viscosity, reduced viscosity and intrinsic viscosity for glucan solutions is determined using capillary viscometer. All viscosity values are calculated by comparing the duration of β-glucan solution flow through a calibrated capillary with the duration of solvent flow (FIG. 8).

Intrinsic viscosity is a molecular mass function, which is described by Mark-Houwink equation:

$$\begin{matrix}[ η ] = {KM}_{α}^{} \\ or & ( 1 ) \\ M = \sqrt[α]{\frac{[ η ]}{K}} & ( 2 )\end{matrix}$$

where:
K and a are empirical constants for a specific polymer;
M—molecular mass of a polymer, (g/mol);
[η]—intrinsic viscosity, (ml/g or 100 ml/g).

As fraction number increases, intrinsic viscosity, and also molar mass of water soluble β-glucans, decrease.

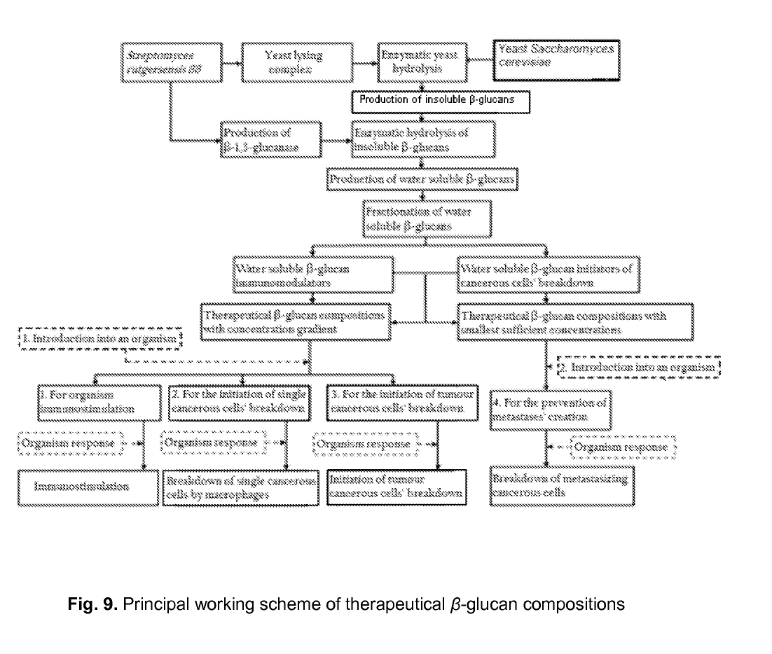

Therapeutical compositions with prepared β-glucan preparations, optimally immunostimulating organism, initiating the breakdown processes of cancerous cells and ensuring the prevention of metastasis formation within healthy tissue, are prepared (FIG. 9).

β-Glucan Compositions, and Their Application for Immunostimulation and the Initiation of Breakdown of Cancerous Cells

Compositions designed to treat cancer in two stages (altogether 26 days) are prepared from β-glucans separated to different fractions.

The first stage of cancer treatment lasts for 16 days. Hereupon, organism is immunostimulated and the breakdown process of cancerous cells is initiated by preparing different compositions from 5 preparations of changeable concentrations (table 1a).

TABLE 1a
Preparation of β-glucan compositions for the first stage
of immunostimulation and breakdown of cancerous cells
Preparation programme

| | Long-chained glucans | | | | Short-chained glucans |
|---|---|---|---|---|---|
| Days the | 5.29 mg/ml-HMM* | 12.92 mg/ml- | 23.03 mg/ml- | 8.76 mg/ml- | 9.6 mg/ml-LMM** |
| preparation | glucans C_{carbohydrates} | C_{carbohydrates} | C_{carbohydrates} | C_{carbohydrates} | glucans C_{carbohydrates} |
| is used | 1 | 2 | 3 | 4 | 5 |
| 1 | 10 mg | | | | |
| | 1.89 ml + 8.11 ml(H₂O) | | | | |
| 2 | 20 mg | | | | |
| | 3.78 ml + 6.22 ml(H₂O) | | | | |
| 3 | | 10 mg | | | |
| | | 0.77 ml + 9.23 ml(H₂O) | | | |
| 4 | | 20 mg | | | |
| | | 1.54 ml + 8.46 ml(H₂O) | | | |
| 5 | | | 10 mg | | |
| | | | 0.43 ml + 9.57 ml(H₂O) | | |
| 6 | | | 20 mg | | |
| | | | 0.86 ml + 9.14 ml(H₂O) | | |
| 7 | | | | 10 mg | |
| | | | | 1.14 ml + 8.86 ml(H₂O) | |
| 8 | | | | 20 mg | |
| | | | | 2.28 ml + 7.72 ml(H₂O) | |
| 9 | | | | | 10 mg |
| | | | | | 1.04 ml + 8.96 ml(H₂O) |
| 10 | | | | | 20 mg |
| | | | | | 2.08 ml + 7.92 ml(H₂O) |

*HMM—higher molecular mass;
**LMM—lower molecular mass.

During the first 10 days, masses of different β-glucan preparation solutions comprising a dose are equal to 10 mg; hence the volumes of different β-glucans and distilled water comprising a dose are calculated respectively according to formulas (3) and (4).

$$\begin{matrix}V ( glucan solution ) = \frac{{m}_{glucans}}{{C}_{preparation}} = \frac{10 mg}{{C}_{total}} & ( 3 ) \\ V ( {H}_{2} {O}_{dist .} ) = V ( total ) - V ( glucan solution ) = 10 ml - V ( glucan solution ) & ( 4 )\end{matrix}$$

*HMM—higher molecular mass; **LMM—lower molecular mass.

During the first 10 days, masses of different β-glucan preparation solutions comprising a dose are equal to 10 mg; hence the volumes of different β-glucans and distilled water comprising a dose are calculated respectively according to formulas (3) and (4).

$$\begin{matrix}V ( glucan solution ) = \frac{{m}_{glucans}}{{C}_{preparation}} = \frac{10 mg}{{C}_{total}} & ( 3 ) \\ V ( {H}_{2} {O}_{dist .} ) = V ( total ) - V ( glucan solution ) = 10 ml - V ( glucan solution ) & ( 4 )\end{matrix}$$

TABLE 1b
Preparation of β-glucan compositions for the first stage
of immunostimulation and cancerous cells' breakdown
Preparation programme

| | Long-chained glucans | | | | Short-chained glucans |
|---|---|---|---|---|---|
| Days the | 5.29 mg/ml-HMM* | 12.92 mg/ml- | 23.03 mg/ml- | 8.76 mg/ml- | 9.6 mg/ml-LMM** |
| preparation | glucans C_{carbohydrates} | C_{carbohydrates} | C_{carbohydrates} | C_{carbohydrates} | glucans C_{carbohydrates} |
| is used | 1 | 2 | 3 | 4 | 5 |
| 11 | 20 mg | | | | 20 mg |
| | 3.78 ml + 6.22 ml(H₂O) | | | | 2.08 ml + 7.92 ml (H₂O) |
| 12 | | 20 mg | | 20 mg | |
| | | 1.54 ml + 8.46 ml(H₂O) | | 2.28 ml + 7.72 ml(H₂O) | |
| 13 | | | 40 mg | | |
| | | | 1.72 ml + 18.28 ml(H₂O) | | |
| 14 | 30 mg | | | | 30 mg |
| | 5.67 ml + 4.33 ml(H₂O) | | | | 3.12 ml + 6.88 ml (H₂O) |
| 15 | | 30 mg | | 30 mg | |
| | | 2.31 ml + 7.69 ml(H₂O) | | 3.42 ml + 6.58 ml(H₂O) | |
| 16 | | | 60 mg | | |
| | | | 5.22 ml + 14.78 ml(H₂O) | | |

*HMM—higher molecular mass;
**LMM—lower molecular mass.

Meanwhile, on the 11-16th treatment days, the amount of different β-glucan preparation solutions comprising a dose are changeable, i.e. equal to 20 mg, 30 mg, 40 mg and 60 mg. Hereupon, the volumes of different β-glucans and distilled water comprising a dose are calculated respectively according to formulas (5) and (6).

$$\begin{matrix}V ( glucan solution ) = \frac{{m}_{glucans}}{{C}_{preparation}} & ( 5 ) \\ V ( {H}_{2} {O}_{dist .} ) = V ( total ) - V ( glucan solution ) = 10 ml - V ( glucan solution ) & ( 6 )\end{matrix}$$

The second stage of cancer treatment lasts for 10 days (days 17-26). Hereupon, organism is affected in order to inhibit the creation of metastases by designing different compositions from 5 preparations with lowest sufficient stable concentrations (table 2).

TABLE 2
Preparation of β-glucan compositions for the second
stage of cancerous cells' metastasizing prevention
Preparation programme

| | Long-chained glucans | | | | Short-chained glucans |
|---|---|---|---|---|---|
| Days the | 5.29 mg/ml-HMM* | 12.92 mg/ml- | 23.03 mg/ml- | 8.76 mg/ml- | 9.6 mg/ml-LMM** |
| preparation | glucans C_{carbohydrates} | C_{carbohydrates} | C_{carbohydrates} | C_{carbohydrates} | glucans C_{carbohydrates} |
| is used | 1 | 2 | 3 | 4 | 5 |

| | | |
|---|---|---|
| 17 | 10 mg | 20 mg |
| | 0.77 ml + 9.23 ml(H₂O) | 0.87 ml + 9.13 ml(H₂O) |
| 18 | 15 mg | 30 mg |
| | 1.16 ml + 8.84 ml(H₂O) | 1.3 ml + 8.7 ml(H₂O) |
| 19 | 10 mg | 20 mg |
| | 0.77 ml + 9.23 ml(H₂O) | 0.87 ml + 9.13 ml(H₂O) |
| 20 | 15 mg | 30 mg |
| | 1.16 ml + 8.84 ml(H₂O) | 1.3 ml + 8.7 ml(H₂O) |
| 21 | 10 mg | 20 mg |
| | 0.77 ml + 9.23 ml(H₂O) | 0.87 ml + 9.13 ml(H₂O) |
| 22 | 15 mg | 30 mg |
| | 1.16 ml + 8.84 ml(H₂O) | 1.3 ml + 8.7 ml(H₂O) |
| 23 | 10 mg | 20 mg |
| | 0.77 ml + 9.23 ml(H₂O) | 0.87 ml + 9.13 ml(H₂O) |
| 24 | 15 mg | 30 mg |
| | 1.16 ml + 8.84 ml(H₂O) | 1.3 ml + 8.7 ml(H₂O) |
| 25 | 10 mg | 20 mg |
| | 0.77 ml + 9.23 ml(H₂O) | 0.87 ml + 9.13 ml(H₂O) |
| 26 | 15 mg | 30 mg |
| | 1.16 ml + 8.84 ml(H₂O) | 1.3 ml + 8.7 ml(H₂O) |

*HMM—higher molecular mass;
**LMM—lower molecular mass.

Meanwhile, on the 17-26th treatment days, the amount of different β-glucan preparation solutions comprising a dose are changeable, i.e. equal to 10 mg, 15 mg, 20 mg and 30 mg. Hereupon, the volumes of different β-glucans and distilled water comprising a dose are also calculated according to formulas (5) and (6).

Organism Response to the Effect of β-Glucan Therapeutical Compositions

Organism immunostimulation. IFN-γ (γ-interferon) increases the antiinfectional and antiviral properties of mammalian immune system, improves phagocytosis and is an important cytokine for immune response. IFN-γ preparations are used for treatment of different diseases. Due to this reason, the alteration in IFN-γ synthesis is used to evaluate the organism immunostimulation effect.

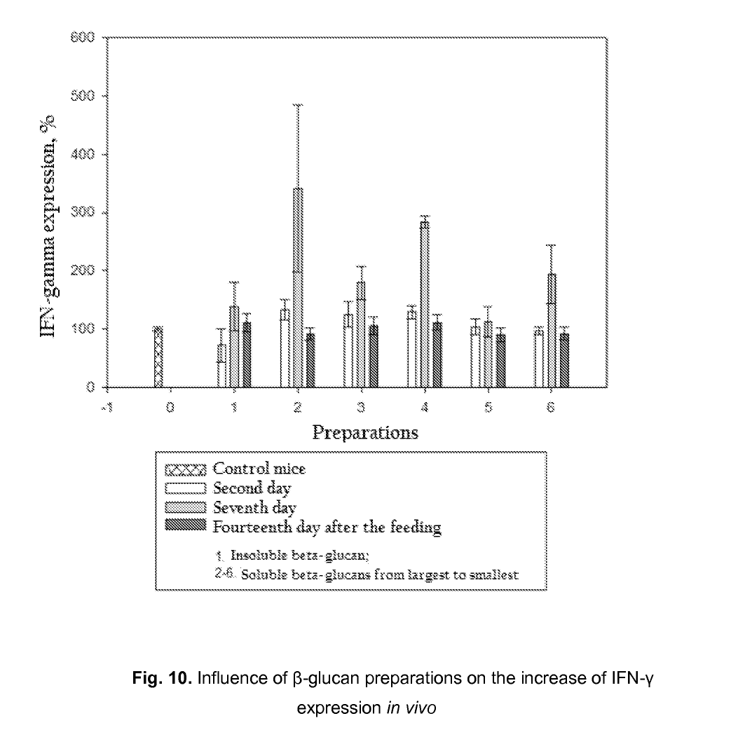

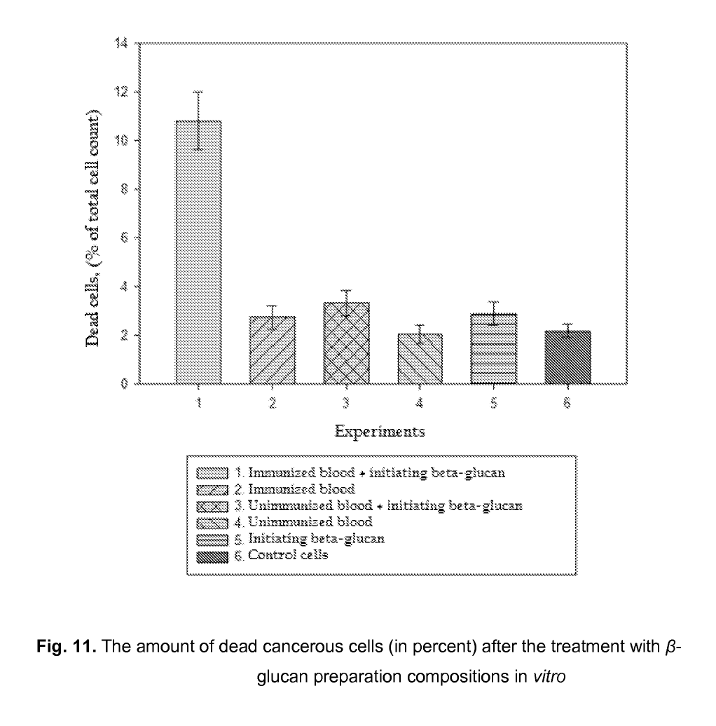

Matured “BALB/c” mice (5 control mice and 5 for each preparation; 35 in total) were fed with different β-glucan preparations (1 insoluble and 5 soluble; 6 in total) for one week. mRNA level in mice blood is monitored during the 2nd and 7th feeding day and 2 weeks after feeding. β-glucan preparations increase IFN-γ synthesis 2-4 times. IFN-γ returns to the initial level when there are no β-glucan preparations in mice rations (FIG. 10).

Initiation of breakdown of cancerous cells. β-glucan preparations have anti-cancer properties. Water soluble β-glucan preparation compositions are used in therapy. “BALB/c” mice are fed for 2 weeks with immunostimulating β-glucans. After the general immunostimulation, mice blood is taken and introduced into cultivation medium for MH22a cancerous mouse cell culture. Additionally, water soluble β-glucan mixture is introduced into MH22a cultivation medium.

The vitality of cancerous cells is analysed after being affected by the mixture for 24 hours. The results show that after affecting MH22a culture cells with immunized mice blood and a mixture of water soluble β-glucans, the amount of dead cancer cells increases by 4-6 times in comparison to:
immunized mice blood;
unimmunized mice blood with water soluble β-glucan mixture;
unimmunized mice blood;
water soluble β-glucan mixture;
cells unaffected by the aforementioned components.

1. A method for obtaining water soluble β-glucan from water insoluble β-glucans, characterized in that the aforementioned method comprises the following steps:
a) isolating of insoluble β-glucan from yeast cell walls using yeast lysing complex and 2M NaOH;
b) specific hydrolysis of insoluble β-glucans using purified β-1,3-glucanase from Streptomyces rutgersensis 88 in order to obtain soluble β-glucans;
c) concentrating of said soluble β-glucans;
d) fractionating of the said soluble β-glucans into immunomodulators and agents initiating' breakdown of cancerous cells;
e) separating of the aforementioned β-glucans into immunomodulators and agents initiating' breakdown of cancerous cells.

2. A method for obtaining water soluble β-glucan from water insoluble β-glucans according to claim 1, characterized in that β-glucans in step b) are specifically hydrolysed to oligosaccharids of a certain molecular mass using β-1,3-glucanase from Streptomyces rutgersensis 88.

3. Soluble β-glucan, characterized in that it is obtained by the method according to claims 1 and 2.

4. Pharmaceutical composition, characterized in that it comprises soluble β-glucans according to claim 3.

5. Pharmaceutical composition according to claim 4, characterized in that it comprises immunomodulators and agents initiating breakdown of cancerous cells.

6. Use of the pharmaceutical composition according to claims 4 and 5, characterized in that it is used for the production of medicine used for human immune system modulation and the breakdown of cancerous cells.

7. Use according to claim 6, characterized in that in order to increase the efficacy of breakdown process on cancerous cells β-glucan fractions of various molecular mass, obtained during specific enzymatic hydrolysis, intrinsic viscosities of which are in a range from 0.3 to 0.01, preferred are from 0.08 to 0.02, are joined together.

8. Use according to claims 6 and 7, characterized in that the immunostimulating preparations and agents initiating breakdown of cancerous cells are introduced alternately.

9. Use according to claim 8, characterized in that agents initiating breakdown of cancerous cells are given to the patient alternately, pending the full degradation of cancerous cells.

10. Use according to any of claims 6-9, characterized in that in order to prevent the adaptation of cancer cell protection systems, agents of small molecular mass initiating breakdown of cancerous cells are used.

11. Use according to any of claims 6-10, characterized in that the said agents are introduced into an organism in vitro, gradually increasing their concentrations.

12. Use according to any of claims 6-11, characterized in that the breakdown of cancerous cells is artificially inhibited, keeping low concentrations of agents initiating breakdown of cancerous cells and high immunostimulation, so that cancerous tissue is degraded slowly, thus preventing the poisoning of an organism with toxic products of breakdown of cancerous cells.

13. Use according to any of claims 6-12, characterized in that the concentrations of immunostimulators and agents initiating' breakdown of cancerous cells is in the range from 0 to 1000 mg/ml during the carrying out of said process.

14. Use according to any of claims 6-13, characterized in that in order to prevent the secondary growth of cancerous cells (metastases) the breakdown process of cancerous cells is repeated by keeping the lowest sufficient stable concentrations of β-glucan compositions initiating breakdown of cancerous cells, depending on treatment efficacy.

15. Pharmaceutical composition of claim 4 or 5, characterized in that it is used for immune system modulation and cancer treatment.
